(19) United States
(12) Patent Application Publication
(10) Pub. No.: US 2025/0319275 A1
(43) Pub. Date: Oct. 16, 2025

(54) MEDICAL OXYGEN HUMIDIFICATION DEVICE

(71) Applicant: TAIPEI MEDICAL UNIVERSITY, Taipei City (TW)

(72) Inventors: Pai-Chien CHOU, Taipei City (TW); Chien-Hua CHEN, Taipei City (TW); Ya-Ting JUANG, Taipei City (TW)

(21) Appl. No.: 19/172,999

(22) Filed: Apr. 8, 2025

(30) Foreign Application Priority Data
Apr. 11, 2024 (TW) 113113576

(51) Int. Cl. A61M 16/10; A61M 16/00; A61M 16/20

(52) U.S. Cl. CPC A61M 16/10; A61M 16/0003; A61M 16/201; A61M 2016/0027; A61M 2202/0007; A61M 2202/0208; A61M 2205/3327; A61M 2205/3331

(57) ABSTRACT

A medical oxygen humidification device includes a container, a semipermeable membrane, a cover, a press plate and a screen. The container includes a bottom wall and a surrounding wall extending upwardly from a periphery of the bottom wall and defining an opening, cooperating with the bottom wall to define a lower space, and formed with upper and lower through holes. The semipermeable membrane covers the opening. The cover removably encloses the lower space, includes a top wall and a peripheral wall surrounding the top wall, and is formed with upper and lower openings. The press plate is movable between the upper and lower openings. The screen is disposed in the lower space, includes a portion spaced apart from the surrounding wall, and closer to the lower through hole than to the upper through hole, and has a number of meshes ranging from 50 to 1000.

### CROSS-REFERENCE TO RELATED APPLICATION

This application claims priority to Taiwanese Invention Patent Application No. 113113576, filed on Apr. 11, 2024, the entire disclosure of which is incorporated by reference herein.

### FIELD

The disclosure relates to a humidifier, and more particularly to a medical oxygen humidification device.

### BACKGROUND

Generally, air inhaled through a nose of a person is heated and humidified by nasal mucosa, and enters an upper respiratory tract into lungs, so the upper respiratory tract may be maintained at a proper temperature and humidity. Once a patient is unable to breathe normally, an endotracheal tube is inserted into a trachea to deliver oxygen to the patient via a respiratory apparatus since oxygen delivery is one of the many support measures for disease treatment. However, pure oxygen delivered by a respiratory apparatus usually has an extremely low humidity level, and is difficult to be sufficiently heated and humidified by the upper
respiratory tract mucosa of the patient, and thus is likely to cause discomfort to the patient.

Referring to FIG. 1, a conventional medical oxygen humidification device 1 includes a container 11 defining an accommodating space for accommodating a predetermined amount of water, a cover 12 covering the accommodating space of the container 11 and including an output passageway 121 and an input passageway 122, a tubular screen 13 configured as a tube, inserted into the input passageway 122 of the cover 12, and extending into the accommodating space of the container 11, and a flow regulator 14 mounted to a top portion of the tubular screen 13 and disposed outside of the accommodating space of the container 11, an oxygen supply fitting 15 connected to the flow regulator 14 and in fluid communication with the input passageway 122 and the tubular screen 13, and a pressure gauge 16 mounted to the oxygen supply fitting 15.

In practical use of the conventional medical oxygen humidification device 1, oxygen is first introduced from the oxygen supply fitting 15 to flow sequentially through the input passageway 122 of the cover 12 and the tubular screen 13 and into the accommodating space to atomize the water in the container 11 into oxygen-containing water vapor such that the atomized oxygen-containing water vapor flows out of the output passageway 121 of the cover 12 and then flows into the lower respiratory tract of the patient.

Although the conventional medical oxygen humidification device 1 may atomize water into oxygen-containing water vapor and supply the same to a patient, it is necessary to introduce oxygen with a relatively high flow rate into the oxygen supply fitting 15 to sufficiently atomize the water. Generally, a flow rate of oxygen must be greater than 6 liters per minute (L/min) to sufficiently atomize water. However, in a case where an amount and a level of the water in the accommodating space of the container 11 are not enough for the oxygen to sufficiently atomize the water for an adequate duration, the oxygen-containing water vapor thus obtained may have a relatively large particle size, which adversely affects the humidifying effect. Therefore, the flow regulator 14 and the pressure gauge 16 are required in the conventional medical oxygen humidification device 1 for monitoring the flow rate and the pressure of the oxygen. Moreover, once the water stays in the container 11 for a period of time, the container 11 may easily become a breeding ground for bacteria, which also adversely affects patient medical treatment.

Another means for humidifying medical oxygen currently used in medical institutions is to utilize an ultrasound humidifying system driven by electricity to aerosolize water into small water droplets which are introduced into the medical oxygen to be supplied to a patient. However, the ultrasound humidifying system consumes electricity, is usually relatively more complicated in structure than the conventional medical oxygen humidification device 1, and also comes with some safety concerns since the ultrasound humidifying system may aerosolize bacteria in the water.

### SUMMARY

Therefore, an object of the present disclosure is to provide a medical oxygen humidification device that can alleviate at least one of the drawbacks of the prior art.

According to an aspect of the disclosure, a medical oxygen humidification device includes a container, a semipermeable membrane, a cover, a press plate and a screen. The container includes a bottom wall and a surrounding wall extending upwardly from a periphery of the bottom wall in a height direction and defining an opening. The bottom wall and the surrounding wall cooperatively define a lower space. The surrounding wall is formed with a lower through hole and an upper through hole that is disposed higher than the lower through hole in the height direction. The semipermeable membrane is disposed on a top edge of the surrounding wall in the height direction for covering the opening. The cover removably encloses the lower space and cooperates with the semipermeable membrane to define an upper space therebetween. The cover includes a top wall and a peripheral wall that surrounds the top wall and that extends downwardly from the top wall in the height direction. The cover is formed with a lower opening and an upper opening that is disposed higher than the lower opening in the height direction. The press plate is disposed in the upper space, is movable upward and downward in the height direction between the lower opening and the upper opening of the cover, and is in contact with an inner surface of the peripheral wall of the cover. The screen is disposed in the lower space, includes a portion spaced apart from the surrounding wall of the container, and closer to the lower through hole than to the upper through hole, and has a number of meshes ranging from 50 to 1000.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a schematic front view of a conventional medical oxygen humidification device.

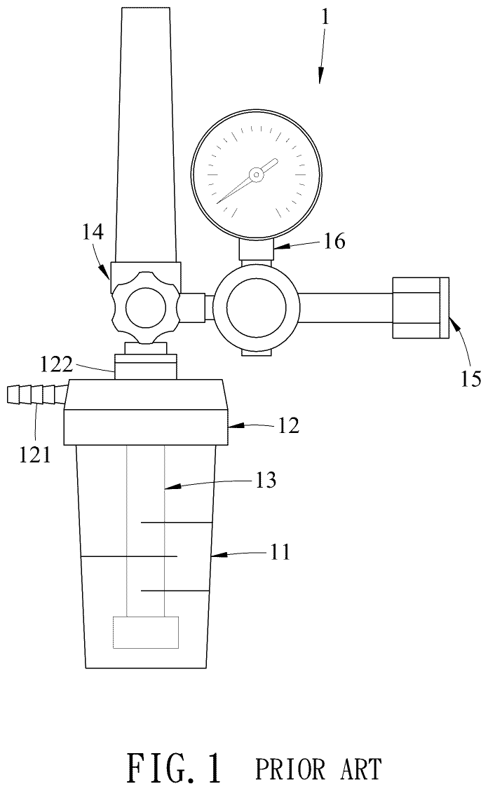

FIG. 2 is a fragmentary sectional view of an embodiment of a medical oxygen humidification device according to the present disclosure.

FIG. 3 is a fragmentary sectional view of the embodiment, illustrating a status of the embodiment when step (a) of a method of using the embodiment is performed.

FIG. 4 is a fragmentary sectional view of the embodiment, illustrating another status of the embodiment subsequent to FIG. 3 when step (a) of the method of using the embodiment is performed.

FIG. 5 is a fragmentary sectional view of the embodiment, illustrating a status of the embodiment when step (b) of the method of using the embodiment is performed.

FIG. 6 is a fragmentary sectional view of the embodiment, illustrating another status of the embodiment subsequent to FIG. 5 when step (b) of the method of using the embodiment is performed.

FIG. 7 is a fragmentary sectional view of the embodiment, illustrating a status of the embodiment when step (c) of the method of using the embodiment is performed.

FIG. 8 is a fragmentary sectional view of the embodiment, illustrating another status of the embodiment subsequent to FIG. 7 when step (c) of the method of using the embodiment is performed.

FIG. 9 is a flow chart of the method of using the embodiment.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

It should be noted herein that for clarity of description, spatially relative terms such as “top,” “bottom,” “upper,” “lower,” “on,” “above,” “over,” “downwardly,” “upwardly” and the like may be used throughout the disclosure while making reference to the features as illustrated in the drawings. The features may be oriented differently (e.g., rotated 90 degrees or at other orientations) and the spatially relative terms used herein may be interpreted accordingly.

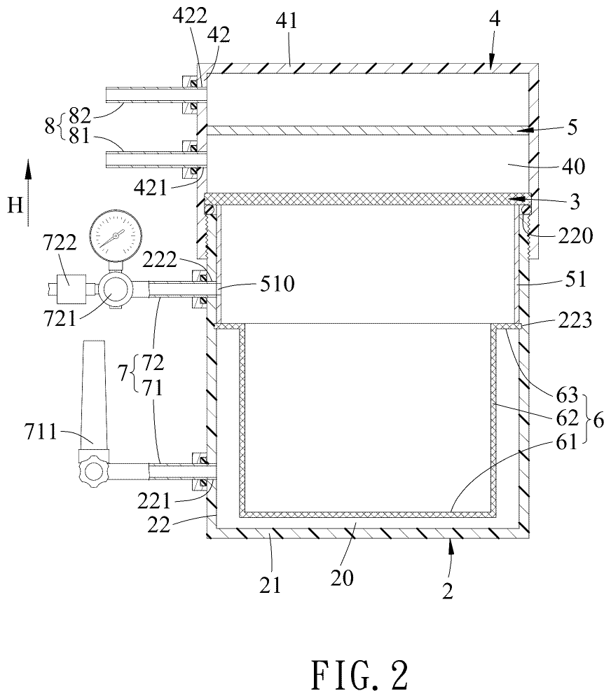

Referring to FIG. 2, an embodiment of a medical oxygen humidification device according to the present disclosure is for humidifying medical oxygen. The medical oxygen humidification device includes a container 2, a semipermeable membrane 3, a cover 4, a press plate 5, a screen 6, a lower duct assembly 7, and an upper pipe assembly 8.

The container 2 includes a bottom wall 21, and a surrounding wall 22 extending upwardly in a height direction (H) from a periphery of the bottom wall 21 and defining an opening. The bottom wall 21 and the surrounding wall 22 cooperatively define a lower space 20. The surrounding wall 22 is formed with a lower through hole 221, and an upper through hole 222 that is disposed higher than the lower through hole 221 in the height direction (H), and that is farther away from the bottom wall 21 than is the lower through hole 221. An inner surface of the surrounding wall 22 of the container 2 is indented toward an outer surface of the surrounding wall 22 to form a groove 223 that is disposed between the lower through hole 221 and the upper through hole 222 in the height direction (H).

The semipermeable membrane 3 is disposed on a top edge 220 of the surrounding wall 22 of the container 2 in the height direction (H) for covering the opening. In this embodiment of the present disclosure, the semipermeable membrane 3 is a reverse osmosis (RO) film that is commercially available on the market.

The cover 4 removably encloses the lower space 20 of the container 2, and cooperates with the semipermeable membrane 3 to define an upper space 40 therebetween. The cover 4 includes a top wall 41, and a peripheral wall 42 surrounding the top wall 41 and extending downwardly from the top wall 41 in the height direction (H). The cover 4 is formed with a lower opening 421, and an upper opening 422 that is disposed higher than the lower opening 421 in the height direction (H) and that is disposed closer to the top wall 41 than is the lower opening 421. In this embodiment, the lower opening 421 and the upper opening 422 are formed in the peripheral wall 42.

In this embodiment, the container 2 and the cover 4 are connected to each other by the following structures. For example, the outer surface of the surrounding wall 22 of the container 2 includes an external thread segment, and an inner surface of the peripheral wall 42 of the cover 4 includes an internal thread segment engaging the external thread segment of the surrounding wall 22 of the container 2. In this embodiment, an O-ring is disposed on the top edge 220 of the surrounding wall 22, such that the cover 4 encloses the lower space 20 of the container 2 through engagement between the external thread segment and the internal thread segment, and the O-ring, but the present disclosure is not limited herein. That is to say, the cover 4 and the container 2 may include other fastening means cooperating with an O-ring for enclosing the lower space 20.

The press plate 5 is disposed in the upper space 40, is movable upward and downward in the height direction (H) between the lower opening 421 and the upper opening 422 of the cover 4, and is in contact with the inner surface of the peripheral wall 42 of the cover 4.

The screen 6 is disposed in the lower space 20, includes a portion spaced apart from the surrounding wall 22 of the container 2 and closer to the lower through hole 221 than to the upper through hole 222, and has a number of meshes ranging from 50 to 1000. Specifically, the screen 6 includes a base wall 61, an annular wall 62 extending upwardly from a periphery of the base wall 61 in the height direction (H) and spaced apart from the surrounding wall 22 of the container 2, and a flange 63 extending from an uppermost end of the annular wall 62 into the surrounding wall 22 of the container 2 and engaging the groove 223. The base wall 61 and the annular wall 62 of the screen 6 are formed with a plurality of micro-through holes extending therethrough.

The lower duct assembly 7 includes a lower duct 71 in spatial communication with the lower through hole 221 of the surrounding wall 22 of the container 2, and an upper duct 72 in spatial communication with the upper through hole 222 of the surrounding wall 22 of the container 2. The upper pipe assembly 8 is disposed higher than the lower duct assembly 7 in the height direction (H), and includes a lower pipe 81 in spatial communication with the lower opening 421 of the cover 4, and an upper pipe 82 in spatial communication with the upper opening 422 of the cover 4.

The embodiment of the medical oxygen humidification device according to the present disclosure further includes an annular support frame 51, a pressure relief unit 711, a pressure sensor 721 and an automatic regulating valve 722. The annular support frame 51 is disposed substantially in the lower space 20, and is disposed between the semipermeable membrane 3 and the flange 63 of the screen 6 in the height direction (H). The annular support frame 51 is formed with a support frame hole 510 extending through the annular support frame 51, disposed at a position adjacent to the upper through hole 222 of the surrounding wall 22 of the container 2, and in spatial communication with the upper through hole 222. The pressure relief unit 711 is mounted to the lower duct 71 of the lower duct assembly 7 and is in spatial communication with a lower duct inner space of the lower duct 71. The pressure sensor 721 and the automatic regulating valve 722 are mounted to the upper duct 72 of the lower duct assembly 7 and are in spatial communication with an upper duct inner space of the upper duct 72. The pressure sensor 721 is configured to detect pressure of fluid in the upper duct inner space of the upper duct 72. The automatic regulating valve 722 is farther from the upper through hole 222 of the container 2 than the pressure sensor 721 is along the upper duct 72. In this embodiment, the pressure relief unit 711 is exemplified using an oxygen flow regulator, but is not limited thereto. Furthermore, the automatic regulating valve 722 includes a regulating valve (not shown) mounted to the upper duct 72 and in spatial communication with the upper duct inner space of the upper duct 72, and a stepper motor (not shown) communicatively connected to the pressure sensor 721 and operable to drive the regulating valve to rotate so as to alter an inner diameter of the upper duct 72.

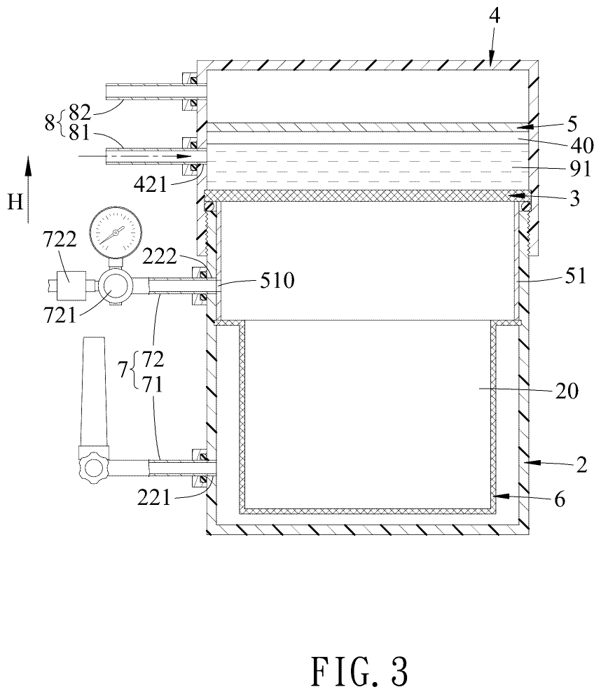

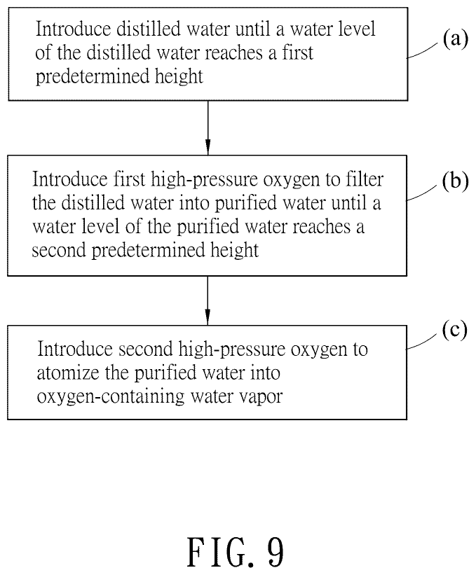

Referring to FIGS. 3 to 9, a method of using the medical oxygen humidification device of the embodiment according to the present disclosure includes step (a), step (b) and step (c) that are performed sequentially.

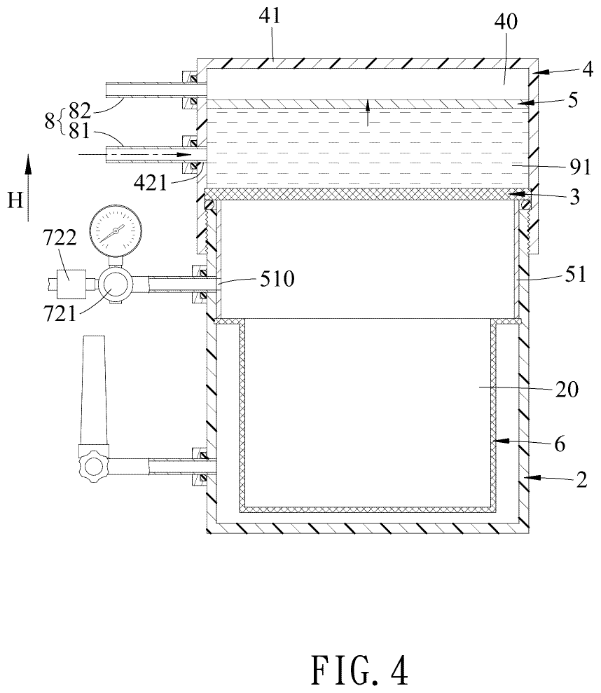

As shown in FIGS. 3 and 4, in step (a), distilled water 91 is first introduced from the lower pipe 81 of the upper pipe assembly 8, and continuously flows into the upper space 40 through the lower opening 421 of the cover 4 until a water level of the distilled water 91 in the upper space 40 reaches a first predetermined height in the height direction (H). In this way, the press plate 5 is moved upwardly (as indicated by an arrow in FIG. 4) in the height direction (H) to a position adjacent to the upper pipe 82 of the upper pipe assembly 8.

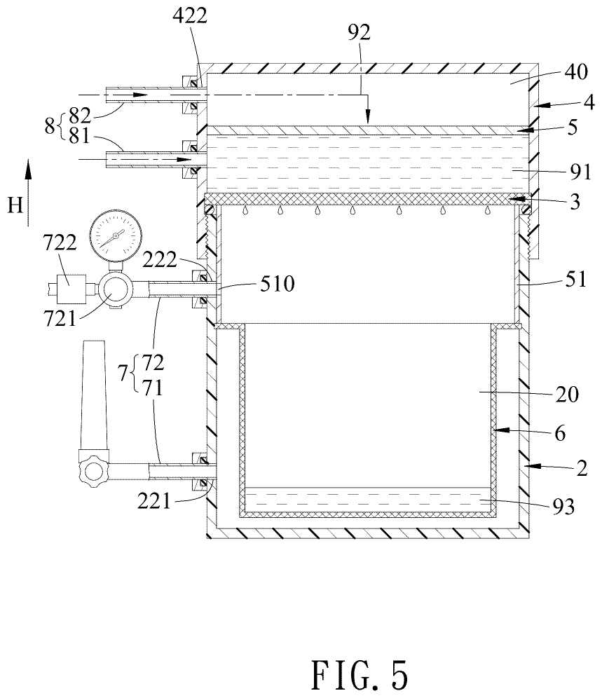

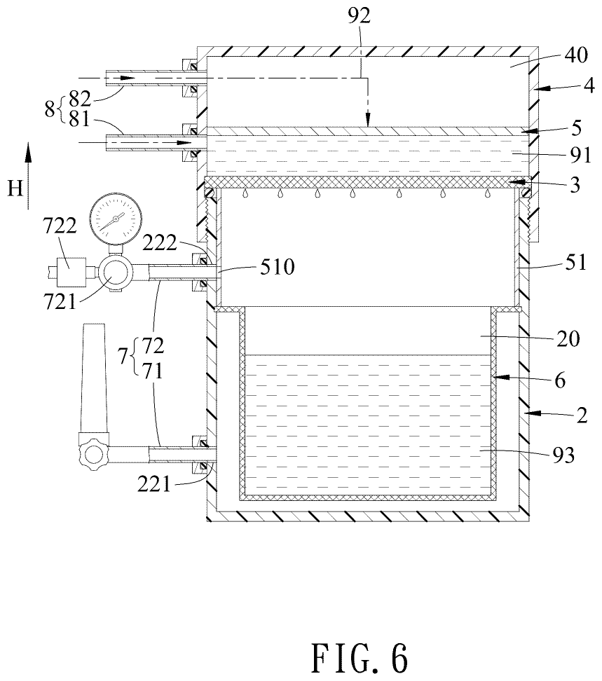

As shown in FIGS. 5 and 6, in step (b), first high-pressure oxygen 92 is introduced from the upper pipe 82 of the upper pipe assembly 8 into the upper space 40 through the upper opening 422 of the cover 4, and the first high-pressure oxygen 92 exerts force on the press plate 5 to move the press plate 5 downwardly, thereby forcing the distilled water 91 in the upper space 40 to penetrate the semipermeable membrane 3 and the distilled water 91 that is filtered by the semipermeable membrane 3, i.e., purified water 93, flows into the lower space 20 and is accumulated on the screen 6 until a water level of the purified water 93 reaches a second predetermined height (see FIG. 6) in the height direction (H). That is to say, introduction of the first high-pressure oxygen 92 into the upper space 40 is stopped after the water level of the purified water 93 reaches the second predetermined height.

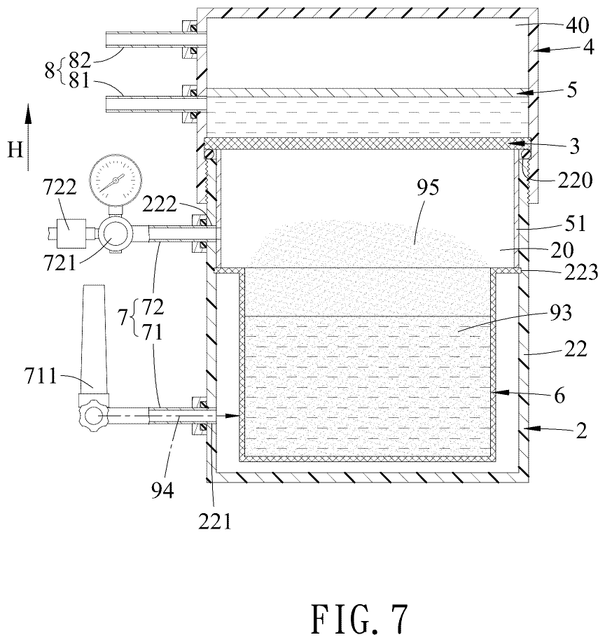

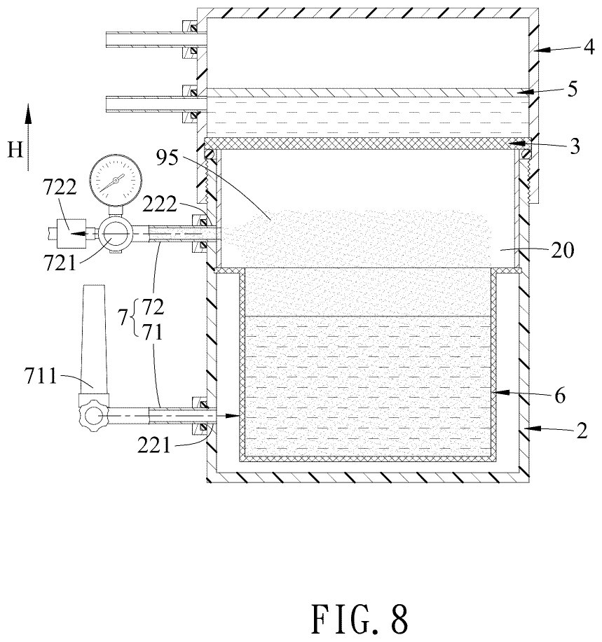

As shown in FIGS. 7 and 8, in step (c), second high-pressure oxygen 94 is introduced into the lower duct 71 of the lower duct assembly 7 and flows into the lower space 20 through the lower through hole 221 of the surrounding wall 22 of the container 2, so that the second high-pressure oxygen 94 passes through the micro-through holes formed in the screen 6 and impacts the purified water 93 in the screen 6 to atomize the purified water 93 into an oxygen-containing water vapor 95 that subsequently flows toward the upper duct 72 of the lower duct assembly 7 (see FIG. 8). Since the oxygen-containing water vapor 95 is atomized from the purified water 93 filtered by the semipermeable membrane 3, the problem of bacteria growth may be alleviated. It should be noted that the pressure relief unit 711 of the embodiment according to the present disclosure is capable of adjusting a flow rate of the second high-pressure oxygen 94 flowing into the lower duct 71 of the lower duct assembly 7 according to pressure of an oxygen supply system of various medical institutions. In this way, damage to the lower duct assembly 7 due to excessive flow rate (i.e., excessive pressure) of the second high-pressure oxygen 94 may be prevented and a possibility of leakage may be reduced. Moreover, by virtue of the pressure sensor 721 that is configured to detect pressure of fluid in the upper duct inner space of the upper duct 72, the pressure in the upper duct 72 may be monitored in real time, thereby ensuring pressure of the oxygen-containing water vapor 95 supplied to the patient is suitable. In a case where the pressure of the oxygen-containing water vapor 95 supplied to the patient is insufficient, the stepper motor (not shown) communicatively connected to the pressure sensor 721 may drive the regulating valve (not shown) to rotate so as to increase the inner diameter of the upper duct 72 and thus increasing the flow rate and the pressure of the oxygen-containing water vapor 95 to meet practical requirements.

It should be noted that the water level of the purified water 93 should be high enough such that there is sufficient time for the purified water 93 to be atomized into the oxygen-containing water vapor 95 to reduce particle sizes of the oxygen-containing water vapor 95 and to increase the humidity of the oxygen-containing water vapor 95. In some embodiments, the second predetermined height is at least higher than one-third of a height of the annular wall 62 of the screen 6 in the height direction (H).

In addition, in a case where pressure of the second high-pressure oxygen 94 is relatively low or a flow rate of the second high-pressure oxygen 94 is relatively low, in order to provide a sufficient humidifying effect, a resistance encountered as the second high-pressure oxygen 94 flows through the screen 6 may be reduced. Specifically, the diameter of each of the micro-through holes of the screen 6 is in negative correlation with the resistance encountered as the second high-pressure oxygen 94 flows through the screen 6. Generally, the screen 6 has a number of meshes ranging from 50 to 1000 (i.e., a diameter of each of the micro-through holes of the screen 6 ranges from 270 μm to 13 μm). In a case where the pressure or the flow rate of the second high-pressure oxygen 94 is insufficient to provide an adequate humidifying effect, the screen 6 may be replaced by another screen 6 that is formed with the micro-through holes each having a greater diameter than that of the screen 6 (i.e., the another screen 6 having a smaller number of meshes) to reduce the resistance encountered as the second high-pressure oxygen 94 flows through the another screen 6, such that the pressure of the second high-pressure oxygen 94 flowing into the lower space 20 through the another screen 6 is increased, thereby improving the humidifying effect. On the other hand, in another case where the pressure or the flow rate of the second high-pressure oxygen 94 is too high, the screen 6 may be replaced by still another screen 6 formed with the micro-through holes each having a smaller diameter than that of the screen 6 (i.e., the still another screen 6 having a larger number of meshes) to increase the resistance encountered as the second high-pressure oxygen 94 flows through the still another screen 6, such that the pressure of the second high-pressure oxygen 94 flowing into the lower space 20 is decreased. In this way, the medical oxygen humidification device of the present disclosure may provide a relatively stable humidifying effect in various medical institutions that provide different pressures or flow rates of oxygen supply. Thus, in actual use of the embodiment according to the present disclosure, even if the pressure or flow rate of the second high-pressure oxygen 94 is too low or too high to provide an appropriate humidifying effect, a medical staff member may simply select the screen 6 with a proper number of meshes to achieve an ideal humidifying effect. In this embodiment, the number of the meshes of the screen 6 ranges from 100 to 800 (i.e., the diameter of each of the micro-through holes of the screen 6 ranges from 150 μm to 18 μm). It should be noted that in still another case where the pressure or the flow rate of the second high-pressure oxygen 94 is sufficient, in order to increase humidification effect, the screen 6 may be replaced by still another screen formed with the micro-through holes each having a smaller diameter than that of the screen 6.

In the following descriptions, some experimental results of the humidifying effect of the embodiment according to the present disclosure are presented. It should be noted herein that the semipermeable membrane 3 is made of polyvinylidene fluoride (PVDF) film in this embodiment but the present disclosure is not limited herein. For example, the PVDF film has a diameter of 47 mm and a diameter of each of the micro-through holes of the screen 6 is 0.22 μm. In the following three experiments, three screens 6 respectively having 100, 400 and 800 meshes are used in their respective experiments. Thus, the humidifying effect on medical oxygen of the embodiment that are achieved using three screens 6 with different numbers of meshes under three different flow rates of oxygen are shown.

Specifically, in each of the three experiments, the volume of the purified water 93 accumulated in the screen 6 is 50 c.c. (see FIG. 6), and the second high-pressure oxygen 94 (as shown in FIG. 7) with three different flow rates is sequentially introduced into the lower duct 71 of the lower duct assembly 7 to perform a three-stage test. A thermo-hygrometer (not shown) is disposed inside the upper duct 72 of the lower duct assembly 7 to measure a temperature and humidity when the three-stage test of each experiment is performed. The three-stage test includes: (1) introducing the second high-pressure oxygen 94 from the lower duct 71 at a flow rate of 1 liter per minute (L/min) for 5 minutes; (2) introducing the second high-pressure oxygen 94 from the lower duct 71 at a flow rate of 5 L/min for 5 minutes; and (3) introducing the second high-pressure oxygen 94 from the lower duct 71 at a flow rate of 10 L/min for 5 minutes.

The medical oxygen humidifying effect of the embodiment according to the present disclosure is shown in Table 1 below.

TABLE 1

| | | | |
|---|---|---|---|
| Flow Rate | Duration | Humidity | Temperature |
| (L/min) | (min) | (%) | (° C.) |

Number of Meshes: 100

| | | | |
|---|---|---|---|
| 1 | 5 | 66.65 | 28.51 |
| 5 | 5 | 60.65 | 28.36 |
| 10 | 5 | 57.05 | 27.63 |

Number of Meshes: 400

| | | | |
|---|---|---|---|
| 1 | 5 | 68.25 | 27.72 |
| 5 | 5 | 59.34 | 27.59 |
| 10 | 5 | 54.18 | 26.50 |

Number of Meshes: 800

| | | | |
|---|---|---|---|
| 1 | 5 | 68.99 | 27.65 |
| 5 | 5 | 64.71 | 27.36 |
| 10 | 5 | 55.53 | 26.52 |

As can be seen from Table 1, in the three experiments with the same flow rate of the second high-pressure oxygen 94, the humidity measured at the upper duct 72 is increased with an increase in the number of meshes of the screen 6 (i.e., decrease in the diameter of each of the micro-through holes). That is to say, the humidity is in positive correlation with the number of meshes of the screen 6. It should be noted that, although the humidity is decreased with an increase in the flow rate of the second high-pressure oxygen 94 in the three experiments in which the screens 6 having 100, 400 and 800 meshes are respectively used, the humidity may be maintained at more than 50%, regardless of whether the flow rate of the second high-pressure oxygen 94 is 1 L/min or 10 L/min. Thus, the humidity may not be drastically affected by the flow rate of the second high-pressure oxygen 94. Additionally, when the number of the meshes of a screen 6 is greater than a certain threshold, e.g., more than 800, the humidity may start to decrease.

In summary, in the embodiment of the medical oxygen humidification device of the present disclosure, since the oxygen-containing water vapor 95 supplied to the patient is atomized from the purified water 93 filtered by the semipermeable membrane 3, the possibility of bacteria growth may be reduced. In addition, the resistance encountered as the second high-pressure oxygen 94 flows through the screen 6 may be adjusted by simply replacing the screen 6 with another screen 6 with a different number of meshes (i.e., the diameter of each micro-through holes). In this way, even if the flow rate of the second high-pressure oxygen 94 is relatively low, humidity of the oxygen-containing water vapor 95 generated by the medical oxygen humidification device of the present disclosure may be maintained at an adequate level for the patient.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A medical oxygen humidification device comprising:
a container that includes a bottom wall and a surrounding wall extending upwardly from a periphery of said bottom wall in a height direction and defining an opening, said bottom wall and said surrounding wall cooperatively defining a lower space, said surrounding wall being formed with a lower through hole and an upper through hole that is disposed higher than said lower through hole in the height direction;
a semipermeable membrane that is disposed on a top edge of said surrounding wall in the height direction for covering said opening;
a cover that removably encloses said lower space and that cooperates with said semipermeable membrane to define an upper space therebetween, said cover including a top wall and a peripheral wall that surrounds said top wall and that extends downwardly from said top wall in the height direction, said cover being formed with a lower opening and an upper opening that is disposed higher than said lower opening in the height direction;
a press plate that is disposed in said upper space, that is movable upward and downward in the height direction between said lower opening and said upper opening of said cover, and that is in contact with an inner surface of said peripheral wall of said cover; and
a screen that is disposed in said lower space, that includes a portion spaced apart from said surrounding wall of said container and closer to said lower through hole than to said upper through hole, and that has a number of meshes ranging from 50 to 1000.

2. The medical oxygen humidification device as claimed in claim 1, wherein the number of meshes of said screen ranges from 100 to 800.

3. The medical oxygen humidification device as claimed in claim 1, wherein:
said medical oxygen humidification device further comprises a lower duct assembly and an upper pipe assembly disposed higher than said lower duct assembly in the height direction;
said lower duct assembly includes a lower duct in spatial communication with said lower through hole of said surrounding wall of said container, and an upper duct in spatial communication with said upper through hole of said surrounding wall of said container; and
said upper pipe assembly includes a lower pipe in spatial communication with said lower opening of said cover, and an upper pipe in spatial communication with said upper opening of said cover.

4. The medical oxygen humidification device as claimed in claim 3, wherein:
said medical oxygen humidification device further comprises a pressure sensor and an automatic regulating valve;
said pressure sensor and said automatic regulating valve are mounted to said upper duct of said lower duct assembly, and are in spatial communication with an upper duct inner space of said upper duct;
said automatic regulating valve is farther from said upper through hole of said surrounding wall of said container than said pressure sensor is along said upper duct.

5. The medical oxygen humidification device as claimed in claim 3, wherein:
said medical oxygen humidification device further comprises a pressure relief unit mounted to said lower duct of said lower duct assembly and in spatial communication with a lower duct inner space of said lower duct.

6. The medical oxygen humidification device as claimed in claim 1, wherein said lower opening and said upper opening of said cover are both formed in said peripheral wall of said cover.

7. The medical oxygen humidification device as claimed in claim 1, wherein:
an inner surface of said surrounding wall of said container is indented toward an outer surface of said surrounding wall to form a groove that is disposed between said lower through hole and said higher through hole in the height direction;
said screen includes a base wall, an annular wall extending upwardly from a periphery of said base wall in the height direction and spaced apart from said surrounding wall of said container, a flange extending from an uppermost end of said annular wall into said surrounding wall and engaging said groove; and
said base wall and said annular wall of said screen are formed with a plurality of micro-through holes extending therethrough.

8. The medical oxygen humidification device as claimed in claim 7, wherein:
said medical oxygen humidification device further comprises an annular support frame disposed substantially in said lower space, and disposed between said semipermeable membrane and said flange of said screen in the height direction; and
said annular support frame is formed with a support frame hole extending through said annular support frame and in spatial communication with said upper through hole of said surrounding wall of said container.
